(19) United States
(12) Patent Application Publication
(10) Pub. No.: US 2021/0008204 A1
(43) Pub. Date: Jan. 14, 2021

(54) COMPOSITIONS AND METHODS OF DETECTING AND TREATING ALZHEIMER'S DISEASE

(71) Applicant: MICROVASCULAR THERAPEUTICS LLC, Tucson, AZ (US)

(72) Inventors: Evan C. Unger, Tucson, AZ (US); Iman Daryaei, Tucson, AZ (US); Emmanuelle Joelle Meuillet, Tucson, AZ (US)

(21) Appl. No.: 16/981,368

(22) PCT Filed: Mar. 28, 2019
(86) PCT No.: PCT/US2019/024713

§ 371 Date: Sep. 16, 2020

Related U.S. Application Data
(60) Provisional application No. 62/650,239, filed on Mar. 29, 2018.

(51) Int. Cl. A61K 41/00; C08K 5/02; A61K 33/16; A61K 47/34; A61K 9/50; A61P 25/28

(52) U.S. Cl. CPC A61K 41/0033; C08K 5/02; A61P 25/28; A61K 47/34; A61K 9/50; A61K 33/16

(57) ABSTRACT

The invention provides microbubbles and/or nanodroplets labeled with diagnostic and/or therapeutic ligands that are useful in the detection and treatment of Alzheimer's disease, or related diseases and conditions, as well as methods of preparation and use thereof.

### PRIORITY CLAIMS AND RELATED PATENT APPLICATIONS

This application claims the benefit of priority to U.S. Provisional Application Ser. No. 62/650,239, filed on Mar. 29, 2018, the entire content of which is incorporated herein by reference in its entirety.

### TECHNICAL FIELDS OF THE INVENTION

This invention relates to pharmaceutical compositions and methods of their preparation and use in diagnosis and therapy. More particularly, the invention relates to microbubbles and/or nanodroplets, and emulsions thereof, labeled with diagnostic and/or therapeutic ligands that are useful in the detection and treatment of Alzheimer's disease, or related diseases and conditions, as well as methods of preparation and use thereof.

### BACKGROUND OF THE INVENTION

Alzheimer's disease (AD) is an irreversible, progressive neurodegenerative disease that slowly destroys memory and thinking skills, and eventually the ability to carry out the simplest tasks. Over 30 million people worldwide suffer from AD. It is currently ranked as the sixth leading cause of death in the United States and accounts for 60% to 70% of cases of dementia. Patients in advanced states of the disease suffer from symptoms can include problems with language, disorientation, withdraw from family and society and other behavioral issues often, leading to eventual loss of bodily functions and ultimately death. A thorough testing and a process involving a series of clinical evaluation and elimination are needed to correctly diagnose AD.

A hallmark of AD is the accumulation of amyloid plaques between nerve cells (neurons) in the brain. Beta-amyloid (or amyloid beta, Aβ) are peptides of 36-43 amino acids that are involved in AD as a main component of the amyloid plaques found in the brains of AD patients. Beta-amyloid is derived from the amyloid precursor protein (APP), which is cleaved by beta secretase and gamma secretase to yield beta amyloid. Beta-amyloid molecules can aggregate to form flexible soluble oligomers which may exist in several forms. Researches have shown that certain misfolded oligomers can induce other beta-amyloid molecules to also take the misfolded oligomeric form, leading to a chain reaction akin to a prion infection. The oligomers are toxic to nerve cells. (Hamley 2012 Chemical Reviews 112 (10): 5147-92; Haass et al. 2007 Nature Reviews Molecular Cell Biology 8 (2): 101-12.)

Another protein implicated in AD is tau protein (or τ proteins), which also forms such prion-like misfolded oligomers. Studies have shown that misfolded beta-amyloid can induce tau to misfold. Pathologies of AD are associated with tau proteins that have become defective and no longer stabilize microtubules properly. (Nussbaum et al. 2013 Prion. 7 (1): 14-9; Pulawski et al. 2012 Applied Biochemistry and Biotechnology 166 (7): 1626-43.)

No medication has been clearly shown to delay or halt the progression of AD. While several medications are currently used to treat the cognitive problems of AD, including acetylcholinesterase inhibitors and the N-Methyl-D-aspartate receptor (NMDA) receptor antagonists, the benefit from their use has been very limited.

Thus, an urgent need and significant challenges remain for novel, safe and reliable diagnostic tools and therapeutic agents for AD.

### SUMMARY OF THE INVENTION

The invention is based in part on the discovery of novel microbubbles and nanodroplets, and emulsions thereof, that are designed to target to beta-amyloid and/or tau protein for improved detection of AD with ultrasound. The invention is also based in part on the discovery of novel microbubbles and/or nanodroplets, and emulsions thereof, that are designed to target to beta-amyloid and/or tau protein for improved treatment of AD with ultrasound. The invention further relates to pharmaceutical compositions and method of preparation and use thereof.

The targeting microbubbles and/or nanodroplets that may be acoustically activated, which bear at least one and preferably two (or more) ligands. The first ligand is a motif that binds to beta-amyloid or to tau protein for detection and localization purposes. The second ligand may comprise a second different ligand and/or an enzyme to degrade beta-amyloid or tau protein. The invention detects and increases the efflux of misfolded and/or aggregated beta-amyloid and or tau protein from the brain, to treat AD.

In one aspect, the invention generally relates to a microscopic bubble or nanoscopic droplet (sometimes referred to as “microscopic or nanoscopic bubble/droplet”) conjugated thereto one or more first ligand having binding affinity to beta-amyloid and one or more second ligand capable of degrading or otherwise metabolizing beta-amyloid.

In another aspect, the invention generally relates to a microscopic or nanoscopic bubble/droplet conjugated thereto one or more first ligand having binding affinity to tau protein and one or more second ligand capable of degrading or otherwise metabolizing tau protein.

In yet another aspect, the invention generally relates to an aqueous emulsion or suspension comprising a microscopic or nanoscopic bubble/droplet disclosed.

In yet another aspect, the invention generally relates to a method for detecting a beta-amyloid. The method includes: administering to a subject in need thereof an aqueous emulsion or suspension comprising a microscopic or nanoscopic bubble/droplet disclosed herein; and imaging a part of the subject to detect the presence of beta-amyloid.

In yet another aspect, the invention generally relates to a method for detecting tau protein. The method includes: administering to a subject in need thereof an aqueous emulsion or suspension comprising a microscopic or nanoscopic bubble/droplet disclosed herein; and imaging a part of the subject to detect the presence of tau protein.

In yet another aspect, the invention generally relates to a method for diagnosing or assessing the risk of Alzheimer's disease. The method includes: administering to a subject in need thereof an aqueous emulsion or suspension comprising a microscopic or nanoscopic bubble/droplet disclosed herein; and imaging a part of the subject to diagnose or assess Alzheimer's disease in the subject.

In yet another aspect, the invention generally relates to a method for treating Alzheimer's disease. The method includes: administering to a subject in need thereof an aqueous emulsion or suspension comprising a microscopic or nanoscopic bubble/droplet disclosed herein; and applying ultrasound to a targeted region of the brain of the subject.

### BRIEF DESCRIPTION OF THE DRAWINGS

FIG. 1. Exemplary chemical structures of ligands that bind to aggregated tau aggregates or AP plaques.

FIG. 2. Exemplary chemical structure of PEGylate phospholipid with reactive functional groups, DSPE-PEGₙ-NHS ester (A) and DSPE-PEGₙ-DBCO.

FIG. 3. Exemplary chemical reaction between ligands with binding affinity to tau aggregates or to AP plaques and phospholipid. Amine group in the small molecules reacts with NETS-Ester group to produce an amide linker (A) and azide group in the small molecule reacts with alkyne group in dibenzylcylcooctyne (DBCO) via cupper-free click chemistry to produce a triazole linker (B).

FIG. 4. Exemplary DSPE-PEGₙ-ligand conjugates are incorporated in the formulation of microbubbles to produce targeting microbubbles for the detection of tau aggregates or Aβ plaques in Alzheimer's disease.

FIG. 5. Exemplary proteins such as Insulin-degrading enzyme (IDE), Neprilysin (NEP), Endothelin-converting enzyme (ECE), Angiotensin converting enzyme (ACE), Plasmin, Matrix metalloprotenases (MMPs), phosphatases, alkaline phosphatases (AP), and antibodies to tau and amyloid beta are conjugated to DSPE-PEGn-NHS ester via lysine or to DSPE-PEGₙ-maleimide to cysteine amino acids in their structure.

FIG. 6. Exemplary DSPE-PEGₙ-ligand and DSPE-PEGₙ-enzyme are incorporated in formulation of microbubbles to produce targeting microbubbles carrying enzyme. Nanoscopic droplets made from MBs localize enzyme in area where tau aggregates or Aβ plaques form, which accelerate degradation and clearance of those proteins.

FIG. 7. Exemplary size analysis of targeted microbubbles.

FIG. 8. Exemplary size analysis of targeted nanodroplets.

FIG. 9. Exemplary data on the effects of the MB, targeted MB and targeted nanodroplets on Tau aggregates. MB for microbubbles alone, MB+US for MB with ultrasound, t2CMB+US for targeted MB with compound 2C and ultrasound. Same for 4C and 4A. t2CND+US for targeted ND with compound 2C. (n=3 samples/condition, bars are the means and error bars are standard errors).

### DETAILED DESCRIPTION OF THE INVENTION

The invention provides novel constructs of micro- and/or nano-bubbles/droplets and emulsions thereof targeted to beta-amyloid and tau protein for superior detection and treatment of Alzheimer's disease with ultrasound.

Ultrasound has been used to open the blood brain barrier. (U.S. Pat. No. 5,752,515.) Microbubbles lower the cavitation thresh-hold and facilitate opening the blood brain barrier. In modes of Alzheimer's disease, microbubbles have been used with ultrasound to open the blood brain barrier and facilitate entry of antibodies to beta-amyloid. (Jordão, et al. 2010 PloS one 5.5, e10549.)

In one aspect, the invention generally relates to a microscopic or nanoscopic bubble/droplet conjugated thereto one or more first ligand having binding affinity to beta-amyloid and one or more second ligand capable of degrading or otherwise metabolizing beta-amyloid.

In another aspect, the invention generally relates to a microscopic or nanoscopic bubble/droplet conjugated thereto one or more first ligand having binding affinity to tau protein and one or more second ligand capable of degrading or otherwise metabolizing tau protein.

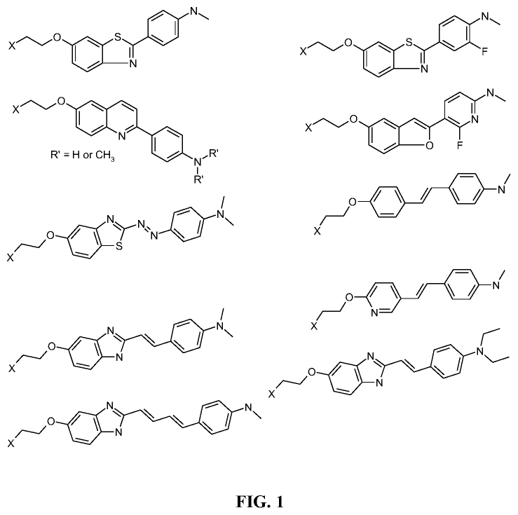

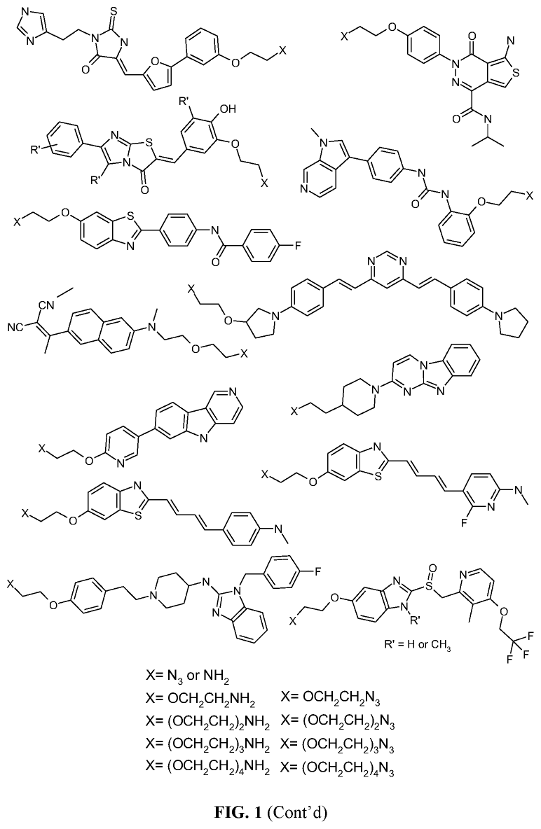

In certain embodiments, the first ligand is a compound, or a derivative thereof, listed in FIG. 1.

In certain embodiments, the second ligand is an enzyme or an antibody, or a fragment thereof.

In certain embodiments, each microscopic or nanoscopic bubble/droplet is conjugated to a plurality of the first ligand.

In certain embodiments, each microscopic or nanoscopic bubble/droplet is conjugated to a plurality of the second ligand.

In certain embodiments, the first ligand is conjugated to the microscopic or nanoscopic bubble/droplet via a PEG linker.

In certain embodiments, the second ligand is conjugated to the microscopic or nanoscopic bubble/droplet via a PEG linker.

In certain embodiments, the microscopic or nanoscopic bubble/droplet is filled with a gaseous and/or liquid material. In certain embodiments, the microscopic or nanoscopic bubble/droplet is filled with a gaseous material. In certain embodiments, the microscopic or nanoscopic bubble/droplet is filled with a liquid material.

In certain embodiments, the gaseous material comprises a fluorinated gas. The term “fluorinated gas”, as used herein, refers to hydrofluorocarbons, which contain hydrogen, fluorine and carbons, or to compounds which contain only carbon and fluorine atoms (also known as perfluorocarbons) and to compounds containing sulfur and fluorine. In the context of the present invention, the term may refer to materials that are comprised of carbon and fluorine or sulfur and fluorine in their molecular structure and are gases at normal temperature and pressure.

In certain embodiments, the fluorinated gas is selected from perfluoromethane, perfluoroethane, perfluoropropane, perfluorocyclopropane, perfluorobutane, perfluorocyclobutane, perfluoropentane, perfluorocylcopentane, perfluorohexane, perfluorocyclohexane, and mixtures of two or more thereof.

In certain embodiments, the fluorinated gas is selected from perfluoropropane, perfluorocyclopropane, perfluorobutane, perfluorocyclobutane, perfluoropentane, perfluorocylcopentane, and mixtures of two or more thereof.

In certain embodiments, the gaseous material further comprises a suitable percentage of non-fluorinated gas or gas mixture, for example, about 2% to about 20% air or nitrogen (e.g., from about 5% to about 20%, from about 10% to about 20%, from about 15% to about 20%, from about 2% to about 15%, from about 2% to about 10%, from about 2% to about 5% of air or nitrogen).

In certain embodiments the fluorocarbon within the microscopic or nanoscopic bubble/droplet exists within the condensed, i.e. liquid state.

In certain embodiments, the microscopic or nanoscopic bubble/droplet is coated by a film-forming material. In certain embodiments, the film-forming material comprises one or more lipids. In certain embodiments, the lipids comprise a phospholipid or a mixture of phospholipids.

Any suitable lipids may be utilized. The lipid chains of the lipids may vary from about 10 to about 24 (e.g., from about 10 to about 20, from about 10 to about 18, from about 12 to about 20, from about 14 to about 20, from about 16 to about 20, 10, 11, 12, 13, 14, 15, 16, 17, 18, 19, 20, 21, 22, 23, 24) carbons in length. More preferably, the chain lengths are from about 16 to about 18 carbons.

In some embodiments, a microscopic or nanoscopic bubble/droplet of the invention is capable of degrading or otherwise metabolizing both of beta-amyloid, tau protein, or both.

In some embodiments, the microscopic or nanoscopic bubble has a diameter in the range of about 10 nm to about 10μm (e.g., from about 10 nm to about 5μm, from about 10 nm to about 1μm, from about 10 nm to about 500 nm, from about 10 nm to about 100 nm, from about 50 nm to about 10μm, from about 100 nm to about 10μm, from about 1μm to about 10μm). In some embodiments, the microscopic or nanoscopic particle or bubble has a diameter from about 10 nm to about 100 nm. In some embodiments, the microscopic or nanoscopic particle or bubble has a diameter from about 100 nm to about 1μm. In some embodiments, the microscopic or nanoscopic particle or bubble has a diameter from about 1μm to about 10μm.

As used herein, the terms “microscopic” and “nanoscopic” refer to microbubble sizes in the micrometer and nanometer ranges, respectively.

In certain embodiments of a method disclosed herein, the microscopic or nanoscopic bubble/droplet have a microscopic size ranging from about 0.5μm to about 10μm (e.g., from about 1μm to about 10μm, from about 2μm to about 10μm, from about 5μm to about 10μm, from about 0.5 p.m to about 5μm, from about 0.5 p.m to about 2μm, from about 1μm to about 5μm).

In certain embodiments of a method disclosed herein, the microscopic or nanoscopic bubble/droplet have a nanoscopic size ranging from about 100 nm to about 800 nm (e.g., from about 100 nm to about 500 nm, from about 100 nm to about 300 nm, from about 120 nm to about 280 nm).

In another aspect, the invention generally relates to an aqueous emulsion or suspension comprising a microscopic or nanoscopic bubble/droplet disclosed.

As used herein, an “emulsion” refers to a heterogeneous system consisting of at least one immiscible liquid dispersed in another in the form of droplets that may vary in size from nanometers to microns. The stability of emulsions varies widely and the time for an emulsion to separate can be from seconds to years. Suspensions may consist of a solid particle or liquid droplet in a bulk liquid phase. As an example, an emulsion of dodecafluoropentane can be prepared with phospholipid or fluorosurfactant and the conjugate incorporated into the emulsion at a ratio of from about 0.1 mole percent to about 1 mole percent or even as much as 5 mole percent, relative to the surfactant used in stabilizing the emulsion.

In certain embodiments, the emulsion or suspension further comprises a pharmaceutically acceptable excipient, carrier, or diluent. Each excipient, carrier, or diluent must be “acceptable” in the sense of being compatible with the other ingredients of the emulsion or suspension and not injurious to the patient. Some examples of materials which can serve as pharmaceutically acceptable excipient, carrier, or diluent include but not limited to normal saline, phosphate buffered saline, propylene glycol, glycerol and polyethylene glycol, e.g. PEG 400 or PEG 3350 MW.

In certain embodiments, the emulsion or suspension is in a homogenized form.

As used herein, a “homogenized” form refers to wherein the emulsion or suspension has been prepared with a form of vigorous mixing. Homogenization can be achieved by any of several processes used to make a mixture of two mutually non-soluble liquids the same throughout. This is generally achieved by turning one of the liquids into a state consisting of extremely small particles distributed uniformly throughout the other liquid. Homogenization is typically conducted using instruments, e.g., an ultra turrax type, an ultrasonic probe mixer/homogenizer, or a high-pressure homogenizer which forces the constituents of the mixture to be emulsified or suspended by forcing them through a small opening or a valve whose interior size can be adjusted, at high pressure.

In yet another aspect, the invention generally relates to a method for detecting a beta-amyloid. The method includes: administering to a subject in need thereof an aqueous emulsion or suspension comprising a microscopic or nanoscopic bubble/droplet disclosed herein; and imaging a part of the subject to detect the presence of beta-amyloid.

In yet another aspect, the invention generally relates to a method for detecting tau protein. The method includes: administering to a subject in need thereof an aqueous emulsion or suspension comprising a microscopic or nanoscopic bubble/droplet disclosed herein; and imaging a part of the subject to detect the presence of tau protein.

In yet another aspect, the invention generally relates to a method for diagnosing or assessing the risk of Alzheimer's disease. The method includes: administering to a subject in need thereof an aqueous emulsion or suspension comprising a microscopic or nanoscopic bubble/droplet disclosed herein; and imaging a part of the subject to diagnose or assess Alzheimer's disease in the subject.

In yet another aspect, the invention generally relates to a method for treating Alzheimer's disease. The method includes: administering to a subject in need thereof an aqueous emulsion or suspension comprising a microscopic or nanoscopic bubble/droplet disclosed herein; and applying ultrasound to a targeted region of the brain of the subject.

In yet another aspect, the invention generally relates to a method for destroying or reducing beta-amyloid aggregates. The method includes: administering to a subject in need thereof an aqueous emulsion or suspension comprising a microscopic bubble and/or nanoscopic droplet disclosed herein; and applying ultrasound to a targeted region of an organ of the subject having beta-amyloid aggregates thereby destroying or reducing the beta-amyloid aggregates.

In yet another aspect, the invention generally relates to a method for destroying or reducing tau protein aggregates. The method includes: administering to a subject in need thereof an aqueous emulsion or suspension comprising a microscopic bubble and/or nanoscopic droplet disclosed herein; and applying ultrasound to a targeted region of an organ of the subject having tau protein aggregates thereby destroying or reducing the tau protein aggregates.

In certain embodiments, the fluorinated gas is selected from perfluoropropane, perfluorocyclopropane, perfluorobutane, perfluorocyclobutane, perfluoropentane, perfluorocylcopentane, and mixtures of two or more thereof. In certain embodiments of a method disclosed herein, the fluorinated gas comprises perfluoropropane, perfluorobutane, or perfluoropentane, or a mixture of two or more thereof.

In certain embodiments of a method disclosed herein, the microscopic or nanoscopic bubble/droplet have a microscopic size ranging from about 0.5μm to about 10μm (e.g., from about 1μm to about 10μm, from about 2μm to about 10μm, from about 5μm to about 10μm, from about 0.5μm to about 5μm, from about 0.5μm to about 2μm, from about 1μm to about 5μm).

In certain embodiments of a method disclosed herein, the microscopic or nanoscopic bubble/droplet have a nanoscopic size ranging from about 100 nm to about 800 nm (e.g., from about 100 nm to about 500 nm, from about 100 nm to about 300 nm, from about 120 nm to about 280 nm).

As used herein, the terms “subject” and “patient” are used interchangeably herein to refer to a living animal (human or non-human). The subject may be a mammal. The terms “mammal” or “mammalian” refer to any animal within the taxonomic classification mammalia. A mammal may be a human or a non-human mammal, for example, dogs, cats, pigs, cows, sheep, goats, horses, rats, and mice. The term “subject” does not preclude individuals that are entirely normal with respect to a disease or condition, or normal in all respects.

As used herein, the terms “treatment” or “treating” a disease or disorder refers to a method of reducing, delaying or ameliorating such a condition, or one or more symptoms of such disease or condition, before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As shown in FIGS. 1-5, one or more ligands are selected that bind to beta-amyloid and/or tau protein. The ligands are attached to a bi-functional spacer, preferably a polyethylene glycol (PEG) group, preferably having a number average molecular weight (MW) in the rage from about 1,000 to about 10,000 Daltons (e.g., from about 2,000 to about 10,000, from about 3,000 to about 10,000 Daltons, from about 4,000 to about 10,000 Daltons, from about 1,000 to about 8,000 Daltons, from about 1,000 to about 6,000 Daltons, from about 3,000 to about 7,000 Daltons, from about 4,000 to about 6,000 Daltons) and more preferably about 5,000 Daltons.

An enzyme or antibody may be used as a second ligand. Preferred enzymes help to metabolize beta-amyloid and/or tau protein. The second ligand is also preferably attached via a bifunctional spacer, preferably a PEG, also with a MW from about 1,000 to about 10,000 Daltons (e.g., from about 2,000 to about 10,000, from about 3,000 to about 10,000 Daltons, from about 1,000 to about 6,000 Daltons, from about 1,000 to about 5,000 Daltons, from about 1,000 to about 4,000 Daltons), more preferably from about 1,000 to about 2,000 Daltons.

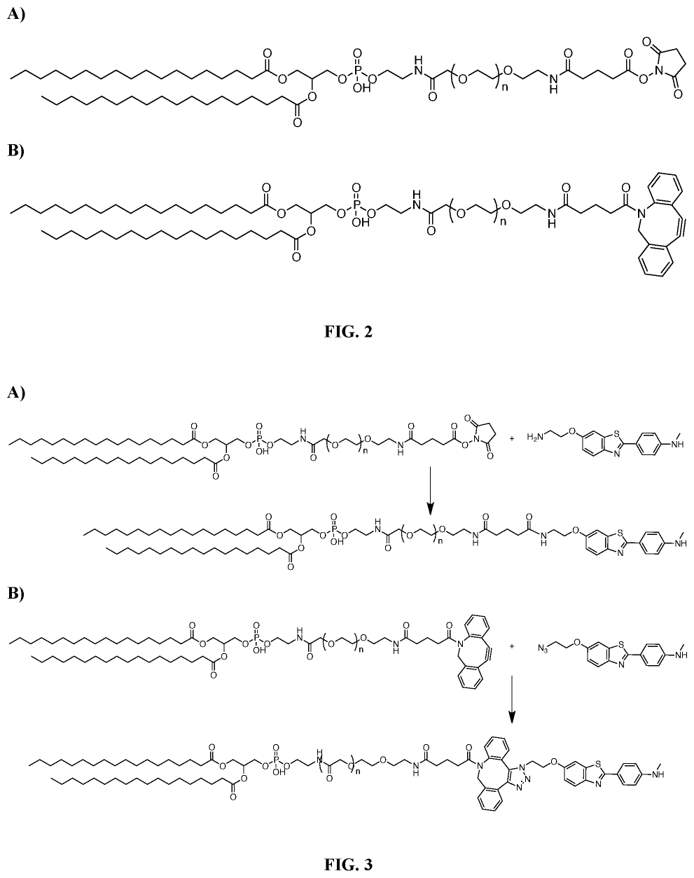

As shown in FIGS. 2-5, the PEG is covalently bound to a lipid anchor, preferably a phospholipid.

In certain embodiments, the phospholipid composition comprises dipalmitoylphosphatidylcholine (“DPPC”), phospholipid 1. DPPC is a zwitterionic compound, and a substantially neutral phospholipid. In certain embodiments, the phospholipid composition comprises a second phospholipid 2 comprising a polyhydroxy head group, and/or a head group of greater than 350 Daltons, having Na⁺, K⁺, Li⁺, and NH₄⁺ counter ions. In certain embodiments, the phospholipid 2 comprises phospholipid 3 comprising a sodium cation and a glycerol head group bound to the phosphoryl moiety. Phospholipid 4 comprises an ammonium counter ion and a polyethylene glycol (“PEG”) head group bound to the phosphoryl moiety. In certain embodiments, the composition comprises a PEG'ylated lipid. In certain embodiments, the MW of the PEG group is from about 1,000 to about 10,000 Daltons. In certain embodiments, the PEG group MW is from about 2,000 to about 5,000 Daltons. In certain embodiments, the PEG group MW is about 5,000 Daltons.

Examples of lipids include phosphoethanolamine-N-[methoxy(polyethylene glycol)-2000] (ammonium salt), 1,2-distearoyl-sn-glycero-3-phosphoethanolamine-N-[methoxy(polyethylene glycol)-2000] (ammonium salt), 1,2-dioleoyl-sn-glycero-3-phosphoethanolamine-N-[methoxy(polyethylene glycol)-2000] (ammonium salt), 1,2-dimyristoyl-sn-glycero-3-phosphoethanolamine-N-[methoxy(polyethylene glycol)-3000] (ammonium salt), 1,2-dipalmitoyl-sn-glycero-3-phosphoethanolamine-N-[methoxy(polyethylene glycol)-3000] (ammonium salt), 1,2-distearoyl-sn-glycero-3-phosphoethanolamine-N-[methoxy(polyethylene glycol)-3000] (ammonium salt), 1,2-dioleoyl-sn-glycero-3-phosphoethanolamine-N-[methoxy(polyethylene glycol)-3000] (ammonium salt), 1,2-dimyristoyl-sn-glycero-3-phosphoethanolamine-N-[methoxy(polyethylene glycol)-5000] (ammonium salt), 1,2-dipalmitoyl-sn-glycero-3-phosphoethanolamine-N-[methoxy(polyethylene glycol)-5000] (ammonium salt), 1,2-distearoyl-sn-glycero-3-phosphoethanolamine-N-[methoxy(polyethylene glycol)-5000] (ammonium salt) and 1,2-dioleoyl-sn-glycero-3-phosphoethanolamine-N-[methoxy(polyethylene glycol)-5000] (ammonium salt). Phospholipid 5 represents dipalmitoylphosphatidylethanolamine, or DPPE. PE, particularly DPPE is a preferred lipid in the invention, preferably in the formulation with the other lipids at concentration of between 5 and 20 mole percent, most preferably 10 mole percent.

Fluorocarbons for use as gaseous precursors in the compositions of the present invention include partially or fully fluorinated carbons, preferably perfluorocarbons that are saturated, unsaturated or cyclic. The preferred perfluorocarbons include, for example, perfluoromethane, perfluoroethane, perfluoropropane, perfluorocyclopropane, perfluorobutane, perfluorocyclobutane, perfluoropentane, perfluorocylcopentane, perfluorohexane, perfluorocyclohexane, and mixtures thereof. More preferably, the perfluorocarbon is perfluorohexane, perfluoropentane, perfluoropropane or perfluorobutane.

### EXAMPLES

### Example 1

### Preparation of Conjugates

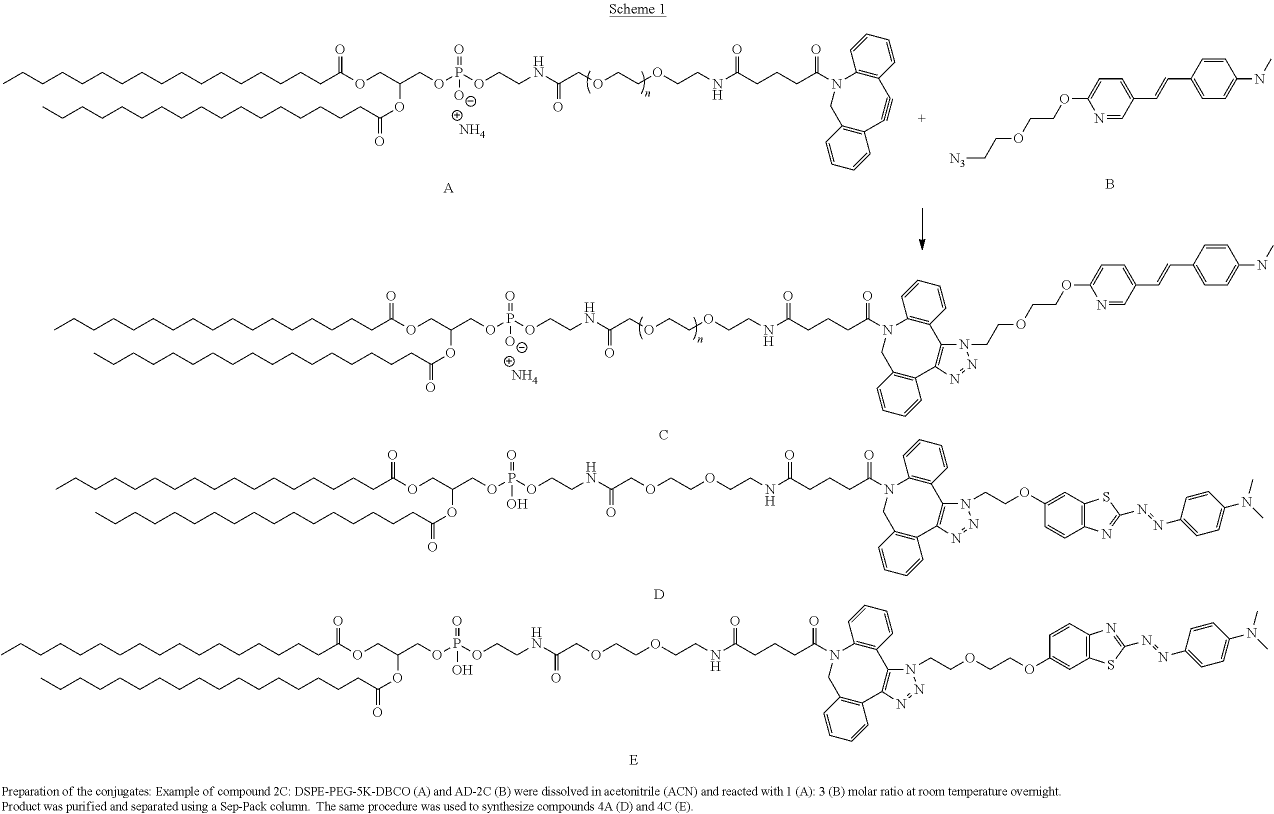

### Preparation of Microbubbles

The biconjugate (1% mol ratio) mixed with DPPC (82% mol ratio), DPPE (10% mol ratio), and DPPE-MPEG-5K (7% mol ratio) separately to produce microbubbles. Phospholipids dissolved in propylene glycol while heating up to 75° C. for 30 min. The solution was added to the salts that were included in the MVT-100 formulation. The final solution distributed in vials (1.5 mL each) and gassed with octafluoropropane (OFP).

### Size Analysis of Microbubbles:

Vials were activated and microbubbles were analyzed for concentration and size distribution:

| | 0.56-1.06 um | | 1.06-2.03 um | | 2.03-5.99 um | | 5.99-10.27 um | |
|---|---|---|---|---|---|---|---|---|
| Example | Conc | | Conc | | Conc | | Conc | |
| Name | (#/mL) | % | (#/mL) | % | (#/mL) | % | (#/mL) | % |
| Sample_AD- | 2.26E+10 | 75.10% | 3.97E+09 | 13.20% | 2.15E+08 | 0.72% | 5.57E+06 | 0.02% |
| 2C_01 | | | | | | | | |
| Sample_AD- | 2.2697E+10 | 74.47% | 4.03E+09 | 13.21% | 2.48E+08 | 0.81% | 5.51E+06 | 0.02% |
| 2C_02 | | | | | | | | |
| Sample_AD- | 1.2247E+10 | 73.74% | 2.49E+09 | 14.97% | 1.62E+08 | 0.97% | 1.61E+06 | 0.01% |
| 2C_03 | | | | | | | | |
| Average | 1.92E+10 | 74.44% | 3.49E+09 | 13.79% | 2.08E+08 | 0.83% | 4.23E+06 | 0.02% |

Exemplary size analysis of targeted microbubbles is shown in FIG. 7.

### Preparation of Nanodroplets

Vials containing the microbubble formulation cooled (−15 to −18° C.) in a cold bath for 3 min. Then the microbubbles were activated and cooled (−15 to −18° C.) in the cold bath for 3 min. Nitrogen (40 to 80 psi) was injected into vials until the milky state of the solution became cloudy. The vials were kept in the cold bath (−15° C. to −18° C.) for 10 min and then they were kept at RT for 1 hr.

### Size Analysis of Nanodroplets

Size analysis of the nanodroplets showed samples with effective diameter of 170 to 250 nm.

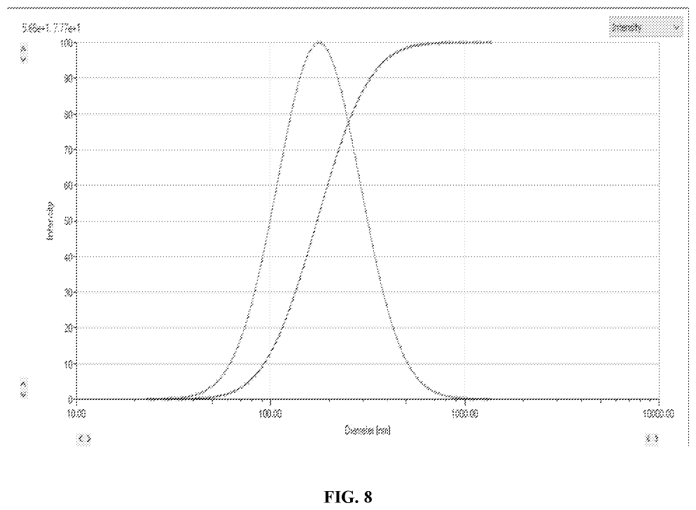

Exemplary size analysis of targeted nanodroplets is shown in FIG. 8.

### Effects of Microbubbles/Nanodroplets on Tau Protein Aggregates in Vitro

Tau proteins form aggregates in the presence of heparin. Fluorescent probes such as Thioflavin T (λ_{excit}=450 nm/λₑₘᵢₛ=480 nm) binds to Tau. A 24-well plate was incubated with the Tau proteins (Tau (K18) P301L mutant pre-formed fibrils and protein monomers; 2 mg/mL) and Heparin 0.03 M in aggregation Tris 20 mM, NaCl 100 mM, EDTA 1 mM buffer pH 7.4 and incubated for 3-4 days at 37° C. in the presence of DTT 1μM.

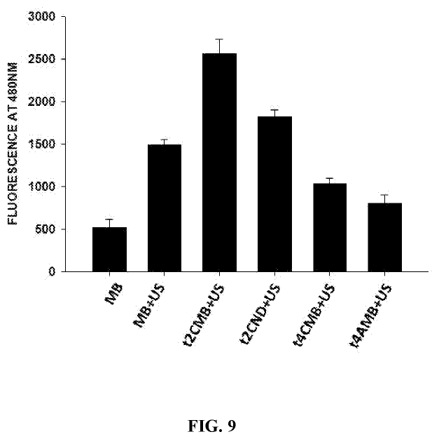

FIG. 9 shows exemplary results on the effects of the microbubble (MB), targeted MB and targeted nanodroplets on Tau aggregates: MB for microbubbles alone, MB + ultrasound (US) for MB with ultrasound, t2CMB+US for targeted MB with compound 2C and ultrasound. Same for 4C and 4A. t2CND+US for targeted ND with compound 2C. (n=3 samples/condition, bars are the means and error bars are standard errors).

Each well received 1.5 mL saline solution and incubated with 200μL microbubbles or nanodroplet for 1 min. The ultrasound conditions applied to each well were the following: 10% duty cycle, 5000 mWatts, frequency at 590 Hz and for 30 sec cycle (Sonic Concepts, TPO-200-02). Following ultrasound application to the wells (or sham application), each content was transferred to an Eppendorf tube and centrifuged for 25 min at 10,000 rpm at room temperature. The liquid phase in the middle was aliquoted for fluorescence measurements at 480 nm in a black ELISA plate (200μL/well). The fluorescence from the protein aggregates was measured as it was released upon the destruction of the aggregates.

The results show that microbubble and ultrasound disrupt the tau aggregates but that microbubble and nanodroplets targeted to Tau cause much greater effects. The in vitro experiments support the concept that ultrasound can be used with tau targeted microbubbles and nanodroplets to treat AD.

### Example 2

See, e.g., U.S. Pat. No. 9,801,959 B2 for detailed descriptions of manufacture of microbubbles.

A blend of lipids was prepared by suspending a mixture of lipids containing DPPC and DPPE-MPEG-5000, DPPE, and DSPE-PEGSK-Conjugate in propylene glycol. The lipids, suspended in propylene glycol, were heated to 70±5° C. until they dissolved. The lipid solution was then added to an aqueous solution containing sodium chloride, phosphate buffer and glycerol and allowed to mix completely by stirring. Each ml of the resultant lipid blend contained 0.75 mg total lipid (consisting of 0.39 mg DPPC, 0.046 mg DPPE, 0.26 mg MPEG-5000-DPPE, and 0.05 mg of DSPE-PEG5K-Conjugate). Each mL of the lipid blend also contained 103.5 mg propylene glycol, 126.2 mg glycerin, 2.34 mg sodium phosphate monobasic monohydrate, 2.16 mg sodium phosphate dibasic heptahydrate, and 4.87 mg sodium chloride in Water for Injection. The pH was 6.2-6.8. One mole percent of the conjugate shown in FIG. 1a was added to the lipid suspension. The material was provided in sealed vials with a headspace containing octafluoropropane (OFP) gas (>80%) with the balance air. Vials were activated using a Vialmix modified dental amalgamator to produce microbubbles targeted to beta-amyloid/tau protein.

### Example 3

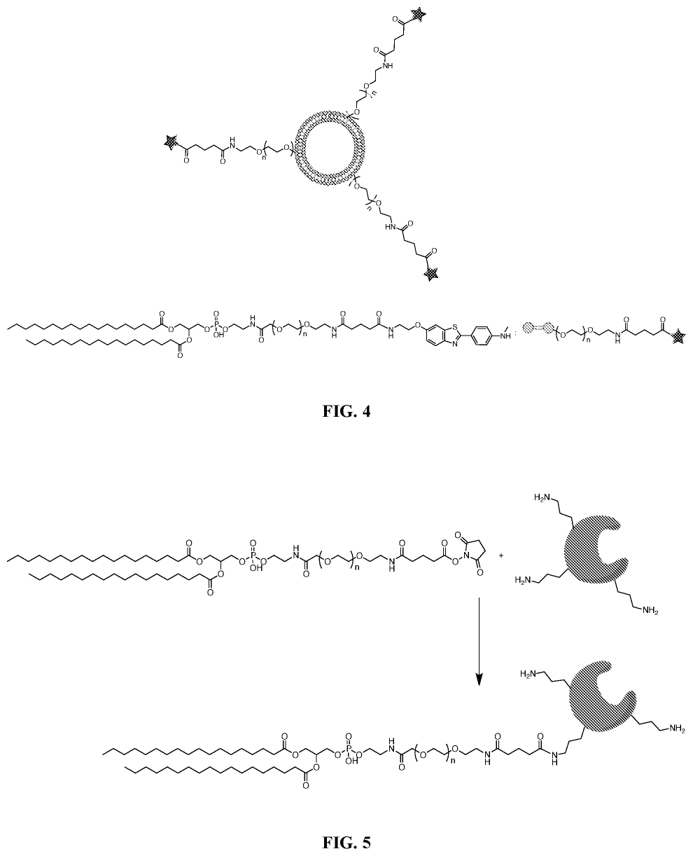

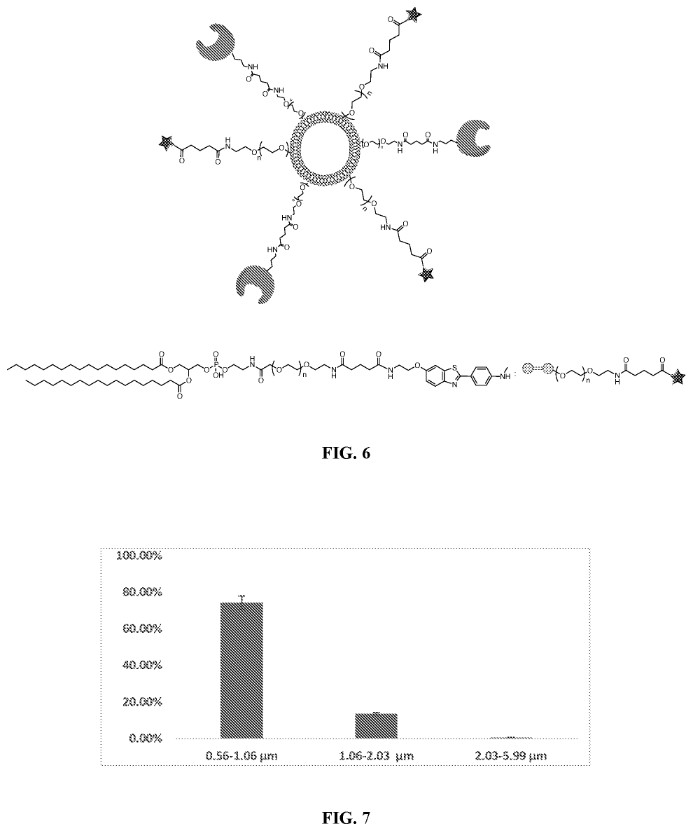

The above was substantially repeated except that separate formulations were prepared using the conjugates shown in FIGS. 4 and 5.

### Example 4

The lipid suspension was prepared as in Example 2 including the conjugate. Microbubbles were formed via agitation by shaking for 45 seconds. The 2 mL vial containing the formed microbubbles was then immersed in a cold bath controlled to a temperature of approximately −15° C. A needle injected nitrogen gas (40-120 psi) into the vial septum. Lipid freezing was avoided by observing the contents of the vial as well as the temperature of the cold bath solution periodically. After pressurizing with a nitrogen gas, the needle was removed from the vial, leaving a pressure head on the solution. The vial was kept in the cold bath for 10-20 min and at room temperature 10-120 min. Particle sizing was performed on the microbubbles as prepared in Example 2 and on the nanodroplets from Example 4. The microbubbles had mean diameter of about 1-2 microns and the nanodroplets had particle size of about 200 nanometers.

### Example 5

Imaging of the brain is performed with PET showing tau deposits beta amyloid aggregation. The nanodroplets from Example 4 are administered intravenously to a patient with AD at a dose of 10×10⁹ nanodroplets and focused ultrasound energy is applied to the brain at 1 Mhz and an MI=1.6. Energy is pulsed at a frequency of 60 Hz. After treatments PET imaging is repeated showing decrease in tau protein deposition.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### Equivalents

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A microscopic or nanoscopic bubble/droplet conjugated thereto one or more first ligand having binding affinity to beta-amyloid and one or more second ligand capable of degrading or otherwise metabolizing beta-amyloid.

2. The microscopic or nanoscopic bubble/droplet of claim 1, wherein the second ligand is an enzyme or an antibody, or a fragment thereof.

3. The microscopic or nanoscopic bubble/droplet of claim or 2, wherein the first ligand is a compound, or a derivative thereof, listed in FIG. 1.

4. The microscopic or nanoscopic bubble/droplet of claim 2, wherein each microscopic or nanoscopic bubble/droplet is conjugated to a plurality of the first ligand.

5. The microscopic or nanoscopic bubble/droplet of claim 4, wherein each microscopic or nanoscopic bubble/droplet is conjugated to a plurality of the second ligand.

6. The microscopic or nanoscopic bubble/droplet of claim 4, wherein the first ligand is conjugated to the microscopic or nanoscopic bubble via a PEG linker.

7. The microscopic or nanoscopic bubble/droplet of claim 5, wherein the second ligand is conjugated to the microscopic or nanoscopic bubble/droplet via a PEG linker.

8. The microscopic or nanoscopic bubble/droplet of claim 5, wherein the microscopic or nanoscopic bubble/droplet is filled with a gaseous material.

9. The microscopic or nanoscopic bubble/droplet of claim 8, wherein the gaseous material comprises a fluorinated gas.

10. The microscopic or nanoscopic bubble/droplet of claim 9, wherein the fluorinated gas is selected from perfluoromethane, perfluoroethane, perfluoropropane, perfluorocyclopropane, perfluorobutane, perfluorocyclobutane, perfluoropentane, perfluorocylcopentane, perfluorohexane, perfluorocyclohexane, and mixtures of two or more thereof.

11. The microscopic or nanoscopic bubble of claim 10, wherein the fluorinated gas is selected from perfluoropropane, perfluorocyclopropane, perfluorobutane, perfluorocyclobutane, perfluoropentane, perfluorocylcopentane, and mixtures of two or more thereof.

12. The microscopic or nanoscopic bubble/droplet of claim 9, being coated by a film-forming material.

13. (canceled)

14. (canceled)

15. The microscopic or nanoscopic bubble/droplet of claim 12, having a microscopic size ranging from about 0.5 to about 10 microns.

16. The microscopic or nanoscopic bubble/droplet of claim 12, having a nanoscopic size ranging from about 120 nm to about 280 nm.

17. A microscopic or nanoscopic bubble/droplet of claim 12, capable of degrading or otherwise metabolizing both of beta-amyloid and tau protein.

18. An aqueous emulsion or suspension comprising a microscopic bubble and/or nanoscopic droplet of claim 12.

19. The emulsion or suspension of claim 18, being in a homogenized form.

20. The emulsion or suspension of claim 18, further comprising a pharmaceutically acceptable excipient, carrier, or diluent.

21-44. (canceled)

45. A method for destroying or reducing beta-amyloid aggregates, comprising:
administering to a subject in need thereof an aqueous emulsion or suspension comprising a microscopic bubble and/or nanoscopic droplet of claim 12; and
applying ultrasound to a targeted region of an organ of the subject having beta-amyloid aggregates thereby destroying or reducing the beta-amyloid aggregates.

46. A method for destroying or reducing tau protein aggregates, comprising:
administering to a subject in need thereof an aqueous emulsion or suspension comprising a microscopic bubble and/or nanoscopic droplet of claim 12; and
applying ultrasound to a targeted region of an organ of the subject having tau protein aggregates thereby destroying or reducing the tau protein aggregates.

47-49. (canceled)
